(19) United States
(12) Patent Application Publication
(10) Pub. No.: US 2024/0157058 A1
(43) Pub. Date: May 16, 2024

(54) THREADED PLUNGER SYRINGES WITH TACTILE FEEDBACK

(71) Applicant: Aspen Neuroscience, Inc., San Diego, CA (US)

(72) Inventor: Edward D. WIRTH, San Diego, CA (US)

(73) Assignee: Aspen Neuroscience, Inc., San Diego, CA (US)

(21) Appl. No.: 18/487,953

(22) Filed: Oct. 16, 2023

Related U.S. Application Data
(60) Provisional application No. 63/416,907, filed on Oct. 17, 2022.

(51) Int. Cl. A61M 5/315

(52) U.S. Cl. CPC A61M 5/3157; A61M 5/31528; A61M 5/31583

(57) ABSTRACT

The present disclosure is directed to threaded plunger syringes with tactile feedback mechanisms and method of using such syringes. The syringes can include a first configuration, wherein the threads of the threaded plunger are engaged by the syringe such that fluid delivery can be precisely controlled, and a second configuration, wherein the threads of the threaded plunger are not engaged such that the syringe can act similar to that of a conventional plunger syringe. In addition, the syringes can include a tactile feedback mechanism that provides tactile feedback when a predetermined amount of fluid volume is dispersed.

### CROSS-REFERENCE TO RELATED APPLICATION

This application claims priority to U.S. provisional application No. 63/416,907, filed Oct. 17, 2022, entitled “THREADED PLUNGER SYRINGES WITH TACTILE FEEDBACK,” the contents of which are incorporated by reference in their entirety for all purposes.

### FIELD

This disclosure relates generally to syringes and methods of using such syringes, and more specifically, to syringes having tactile feedback mechanisms and methods of using such syringes.

### BACKGROUND

Methods for delivering cells in some contexts require precise control of delivery, including with small volumes. For instance, delivery of neuronally differentiated cells, such as cells differentiated to floor plate midbrain progenitor cells or determined dopamine (DA) neurons, involves controlled delivery to the brain tissue, such as to the striatum. Existing devices are not suitable in all respects for use in such cell therapy applications. Provided herein are devices that address such needs.

### SUMMARY

Described herein are threaded plunger syringes capable of providing tactile feedback to the operator so that the operator knows exactly how much fluid is being dispersed from the syringe during use. Specifically, the tactile feedback mechanism can provide tactile feedback for each predetermined amount of fluid volume dispersed. For example, the syringes disclosed herein can provide tactile feedback at every microliter dispersed.

In addition, the syringes described herein can have a first configuration, wherein the threads of the threaded plunger are engaged by the syringe such that fluid delivery can be precisely controlled. The syringes can also have a second configuration, wherein the threads of the threaded plunger are not engaged such that the syringe can act similar to that of a conventional syringe. For example, in the second configuration, fluid can be drawn in manually into the syringe without the user of the threads of the threaded plunger.

In some embodiments, the syringes described herein can be metered syringes that can facilitate slow injection of small volumes of fluid (e.g., investigational drugs including cell therapies), which cannot easily be done with conventional syringes that utilize manual depression of the syringe plunger. In some embodiments, the fluid in the syringes can be cell suspensions (e.g., stem cells or cells differentiated from stem cells). In some embodiments, the syringes disclosed herein can be used for a medical procedure or treatment where the precise volume (e.g., one microliter) of fluid (e.g., stem cells or cells differentiated from stem cells) disbursement from the syringe can be critical. The syringes disclosed herein can also provide feedback to the user when such precise volume has been disbursed.

In some embodiments, the syringes described herein can be designed and/or manufactured in alignment with ISO 13485 and FDA Quality System Regulations. In some embodiments, the various components described below of the syringes can be manufactured of materials that are biocompatible (e.g., silicone that is USP Class VI), inert, DMSO compatible, shatter resistant, and/or compatible with use in an MRI scanner (non-magnetic). In some embodiments, the fluid path components (e.g., syringe tip, syringe body, plunger tip described below) of the syringes disclosed herein can be manufactured of materials that are biocompatible (e.g., silicone that is USP Class VI), inert, DMSO compatible, shatter resistant, and/or compatible with use in an MRI scanner (non-magnetic). In some embodiments, the non-fluid path components (e.g., components other than syringe tip, syringe body, and plunger tip) of the syringes disclosed herein can be manufactured of materials that are shatter resistant and/or compatible with use in an MRI scanner (non-magnetic). In some embodiments, the syringes disclosed herein can be single-use and are designed to interface with an industry standard Luer Lock connection to off-the-shelf needles, Luer Lock caps, and cannulas.

In some embodiments, a syringe includes a syringe body having a proximal end and a distal end; a threaded plunger, wherein a portion of the threaded plunger is within the syringe body; a syringe body mount connected to the proximal end of the syringe body, wherein the syringe body mount comprises: a control base fixed to the proximal end of the syringe body; and a control knob rotatably connected to the control base, wherein the control knob is configured to engage with threads of the threaded plunger such that rotation of the control knob with respect to the control base causes the threaded plunger to move longitudinally in the syringe body, wherein the syringe body mount is configured to provide tactile feedback to an operator of the syringe when a predetermined amount of fluid volume is dispersed from the syringe.

In some embodiments, the control knob comprises a plunger disengagement mechanism that disengages the control knob from the threads of the threaded plunger such that the threaded plunger is configured to move longitudinally in the syringe body without rotation of the control knob. In some embodiments, the plunger disengagement mechanism comprises at least one depressible button. In some embodiments, the control knob comprises a spring configured to cause the at least one depressible button to engage with the threads of the threaded plunger and disengage with the threads of the threaded plunger when the at least one depressible button is depressed. In some embodiments, the plunger disengagement mechanism comprises two depressible buttons. In some embodiments, the control knob comprises an O-ring configured to cause the two depressible buttons to engage with the threads of the threaded plunger and disengage with the threads of the threaded plunger when the two depressible buttons are depressed. In some embodiments, the control knob is configured to disengage with the threads of the threaded plunger such that the threaded plunger is configured to move longitudinally in the syringe body without rotation of the control knob. In some embodiments, the control knob is configured to disengage with the threads of the threaded plunger such that the threaded plunger is configured to move longitudinally in the syringe body via pulling or pushing on a plunger handle on a proximal end of the threaded plunger. In some embodiments, the control knob is configured to engage with the threads of the threaded plunger such that clockwise rotation of the control knob with respect to the control base causes the threaded plunger to move longitudinally towards the distal end of the syringe body and counterclockwise rotation of the control knob with respect to the control base causes the threaded plunger to move longitudinally towards the proximal end of the syringe body. In some embodiments, the control base comprises a ball plunger and the control knob comprises a plurality of detents such that as the control knob rotates, the ball plunger is depressed between the control base and control knob until each detent where the ball plunger fits into the detent providing tactile feedback to the operator. In some embodiments, the distance between the plurality of detents corresponds to the predetermined amount of fluid volume. In some embodiments, the control base comprises a flexure and the control knob comprises a plurality of detents such that as the control knob rotates, the flexure experiences resistance until each detent where the flexure relaxes providing tactile feedback to the operator. In some embodiments, the distance between the plurality of detents corresponds to the predetermined amount of fluid volume. In some embodiments, the distal end of the syringe body comprises a Luer Lock connector. In some embodiments, the syringe includes a plunger handle on a proximal end of the threaded plunger and a plunger tip on a distal end of the threaded plunger. In some embodiments, the syringe includes a syringe tip comprising a recess configured to receive at least a portion of the syringe body and a channel configured to receive at least a portion of the plunger tip. In some embodiments, the channel comprises a first proximal portion and a second distal portion and the first proximal portion is configured to receive at least a portion of the plunger tip. In some embodiments, the threaded plunger is a left-hand threaded plunger. In some embodiments, the predetermined amount of fluid volume is 1 microliter. In some embodiments, the syringe body mount is configured to provide audio or visual feedback to an operator of the syringe for a predetermined amount of fluid volume dispersed from the syringe. In some embodiments, the syringe body has a working volume of 450-550 microliters. In some embodiments, the threaded plunger does not rotate while moving longitudinally in the syringe body. In some embodiments, the distal end of the syringe is connected to a cannula.

In some embodiments, a method of treating a subject having a disease or condition includes using any syringe disclosed herein to deliver a cell suspension to the subject. In some embodiments, the disease or condition is a neurodegenerative disease. In some embodiments, the disease or condition is a Lewy body disease (LBD). In some embodiments, the disease or condition is Parkinson's disease. In some embodiments, the cell suspension comprises stem cells or cells that have been differentiated from stem cells. In some embodiments, the cell suspension comprises neurally differentiated cells. In some embodiments, the cell suspension is delivered to the brain tissue of the subject. In some embodiments, the cell suspension is delivered to the striatum, optionally the putamen, of the subject. In some embodiments, the subject is a human. In some embodiments, a cannula is connected to the syringe to deliver the cell suspension to the subject.

It will be appreciated that any of the variations, aspects, features and options described in view of the syringes apply equally to the systems, methods, other syringes, and vice versa. It will also be clear that any one or more of the above variations, aspects, features and options can be combined.

Additional advantages will be readily apparent to those skilled in the art from the following detailed description. The aspects and descriptions herein are to be regarded as illustrative in nature and not restrictive.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### BRIEF DESCRIPTION OF THE FIGURES

Embodiments will now be described, by way of example only, with reference to the accompanying drawings, in which:

FIG. 1A is an image of a Hamilton Company syringe Model 1001, PN 81341.

FIG. 1B is an image of the Hamilton Company syringe Model 1001, PN 81341 with the syringe plunger nut disengaged from the syringe body mount.

FIG. 2A illustrates an exemplary threaded plunger syringe in accordance with some embodiments disclosed herein.

FIG. 2B illustrates an exemplary cross section of section A-A of the syringe of FIG. 2A in accordance with some embodiments disclosed herein.

FIG. 3 illustrates an exemplary cross section of a syringe body mount in accordance with some embodiments disclosed herein.

FIG. 4 illustrates an exemplary cross section of a control knob in accordance with some embodiments disclosed herein.

FIG. 5 illustrates another exemplary cross section of a syringe body mount in accordance with some embodiments disclosed herein.

FIG. 6 illustrates an exemplary cutaway of a control knob in accordance with some embodiments disclosed herein.

FIG. 7 illustrates an exemplary top view of a control base in accordance with some embodiments disclosed herein.

FIG. 8 illustrates an exemplary plunger disengagement mechanism in accordance with some embodiments disclosed herein.

FIG. 9 illustrates another exemplary cross section of a control knob in accordance with some embodiments disclosed herein.

FIG. 10A illustrates an exemplary cross section of a control knob when the control knob is engaged with the threaded plunger in accordance with some embodiments disclosed herein.

FIG. 10B illustrates an exemplary cross section of a control knob when the control knob is disengaged with the threaded plunger in accordance with some embodiments disclosed herein.

FIG. 11 illustrates an exemplary cross section of a control base in accordance with some embodiments disclosed herein.

FIG. 12 illustrates an exemplary view of the underside of a control knob in accordance with some embodiments disclosed herein.

FIG. 13A illustrates an exemplary cross section of a syringe body mount with the ball plunger in a detent in accordance with some embodiments disclosed herein.

FIG. 13B illustrates an exemplary cross section of a syringe body mount with the ball plunger depressed between the control base and the control knob in accordance with some embodiments disclosed herein.

FIG. 13C illustrates another exemplary cross section of a syringe body mount with the ball plunger in a detent in accordance with some embodiments disclosed herein.

FIG. 14A illustrates a front view of another exemplary threaded plunger syringe in accordance with some embodiments disclosed herein.

FIG. 14B illustrates a side view of an exemplary threaded plunger syringe in accordance with some embodiments disclosed herein.

FIG. 14C illustrates an exemplary cross section of section A-A of the syringe of FIG. 14B in accordance with some embodiments disclosed herein.

FIG. 14D illustrates a top view of an exemplary threaded plunger syringe in accordance with some embodiments disclosed herein.

FIG. 14E illustrates an exemplary cross section of section B-B of the syringe of FIG. 14B in accordance with some embodiments disclosed herein.

FIG. 15 illustrates an exploded view of the main components of an exemplary threaded plunger syringe in accordance with some embodiments disclosed herein.

FIG. 16 illustrates an exploded view of various components of an exemplary threaded plunger syringe in accordance with some embodiments disclosed herein.

FIG. 17 illustrates a more detailed exploded view of various components of an exemplary threaded plunger syringe in accordance with some embodiments disclosed herein.

FIG. 18 illustrates an exemplary cutaway view of a syringe body mount of in accordance with some embodiments disclosed herein.

FIG. 19A illustrates an exemplary internal view of a control knob when the control knob is engaged with the threaded plunger in accordance with some embodiments disclosed herein.

FIG. 19B illustrates an exemplary internal view of a control knob when the control knob is disengaged with the threaded plunger in accordance with some embodiments disclosed herein.

FIG. 20A illustrates a front view of another exemplary threaded plunger syringe in accordance with some embodiments disclosed herein.

FIG. 20B illustrates a side view of an exemplary threaded plunger syringe in accordance with some embodiments disclosed herein.

FIG. 21 illustrates an exploded view of various components of an exemplary threaded plunger syringe in accordance with some embodiments disclosed herein.

FIG. 22 illustrates an example of a detent holder in accordance with some embodiments disclosed herein.

FIG. 23 illustrates an example of a syringe body mount in accordance with some embodiments disclosed herein.

FIG. 24A illustrates a front view of another exemplary threaded plunger syringe in accordance with some embodiments disclosed herein.

FIG. 24B illustrates an exemplary cross section of section A-A of the syringe of FIG. 24A in accordance with some embodiments disclosed herein.

FIG. 24C illustrates an exemplary enlarged section of the circled section B of the syringe of FIG. 24B in accordance with some embodiments disclosed herein.

FIG. 25A illustrates an example of a syringe tip in accordance with some embodiments disclosed herein.

FIG. 25B illustrates an exemplary side view of a syringe tip in accordance with some embodiments disclosed herein.

FIG. 25C illustrates an exemplary cross section of section A-A of the syringe tip of FIG. 25B in accordance with some embodiments disclosed herein.

In the Figures, like reference numbers correspond to like components of the syringes unless otherwise stated.

### DETAILED DESCRIPTION

Reference will now be made in detail to implementations and embodiments of various aspects and variations of syringes, systems, and methods described herein. Although several exemplary variations of the syringes, systems, and methods are described herein, other variations of the syringes, systems, and methods may include aspects of the syringes, systems, and methods described herein combined in any suitable manner having combinations of all or some of the aspects described.

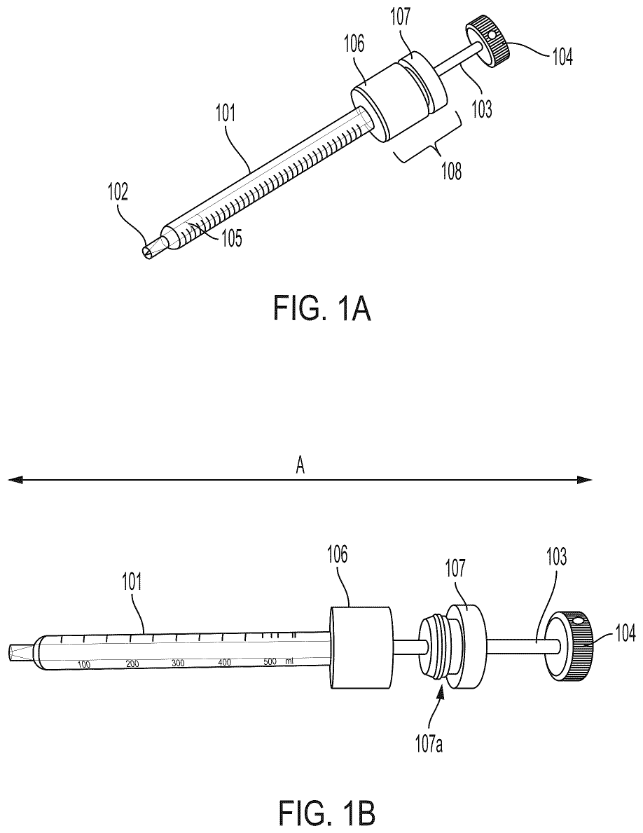

A typical threaded plunger syringe used for microliter volume disbursements is the Hamilton Company syringe Model 1001, PN 81341, shown in FIGS. 1A and 1B. The Hamilton Company syringe has a glass syringe body 101 with a syringe tip 102 at the distal end of the syringe body and a syringe body mount 106 connected to the proximal end of the syringe body. When the syringe plunger nut 107 is engaged (via threads 107a) with the syringe body mount to form the plunger engagement mechanism 108, a user of the syringe can rotate the plunger handle 104 of the threaded plunger 103 to cause the threaded plunger to move longitudinally in the syringe body. Rotation in the clockwise direction of the threaded plunger can cause the threaded plunger to move longitudinally toward the distal end of the syringe body and thereby cause the plunger tip 105 to dispense fluid out the syringe tip. In contrast, rotation of the threaded plunger in the counterclockwise direction causes the threaded plunger to move longitudinally toward the proximal end of the syringe body. The longitudinal axis of the syringe is the axis running lengthwise of the syringe and is shown as axis A in FIG. 1B.

While rotating the plunger handle, there is no feedback from the syringe for a user to know how much fluid has been dispensed during rotation of the threaded plunger. In addition, in order to fill the syringe body with fluid, the syringe plunger nut needs to be unscrewed or disengaged from the syringe body mount as shown in FIG. 1B. Once the syringe plunger nut is disengaged from the syringe body, the threaded plunger can move longitudinally in the syringe body without rotation of the threaded plunger. As such, an operator of the Hamilton Company syringe can fill the syringe by pulling on the threaded plunger handle to draw fluid into the syringe. However, disengaging the syringe plunger nut from the syringe body mount can be cumbersome as well as completely detach the syringe plunger nut (and threaded plunger) from the syringe body.

Described herein are threaded plunger syringes capable of providing tactile feedback to the operator so that the operator knows exactly how much fluid is being dispersed from the syringe during use. Specifically, the tactile feedback mechanism can provide tactile feedback for each predetermined amount of fluid volume dispersed. For example, the syringes disclosed herein can provide tactile feedback at every microliter dispersed.

In addition, the syringes described herein can have a first configuration, wherein the threads of the threaded plunger are engaged by the syringe such that fluid delivery can be precisely controlled, and a second configuration, wherein the threads of the threaded plunger are not engaged such that the syringe can act similar to that of a conventional plunger syringe. The threaded plunger syringes described herein can be used for various purposes and/or procedures. In some embodiments, the threaded plunger syringes can be used for medical procedures (e.g., neurological procedures). In some embodiments, the threaded plunger syringes can facilitate the administration of a cell suspension or drug product. In some embodiments, the threaded plunger syringes can facilitate administration of a cell suspension or drug product at a precisely user-controlled volumetric flowrate with tactile feedback.

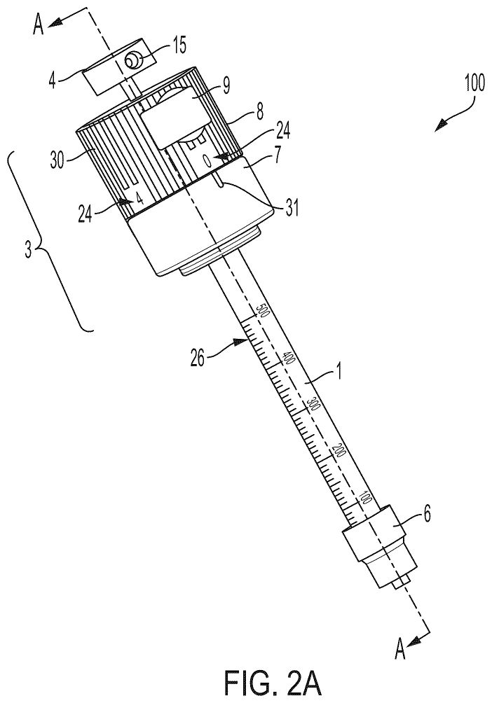

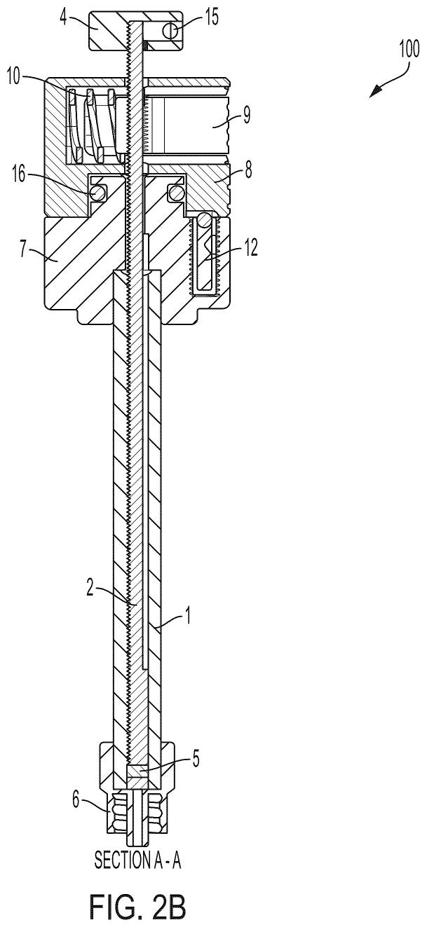

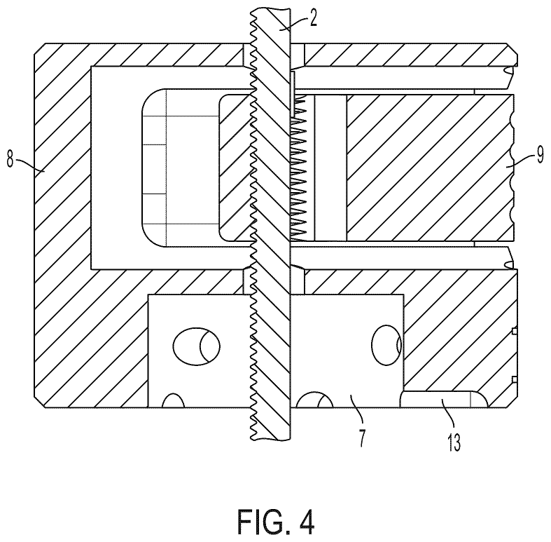

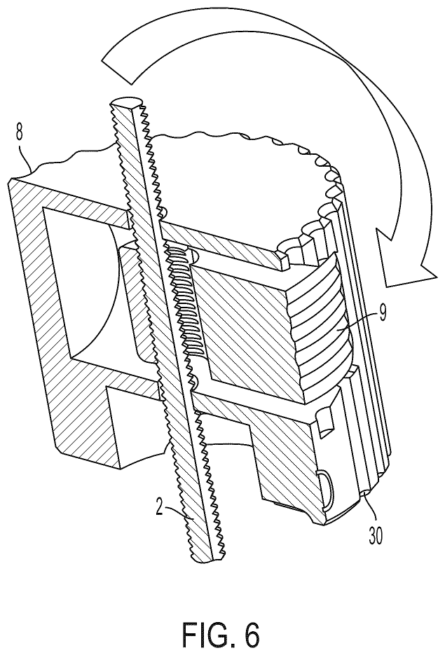

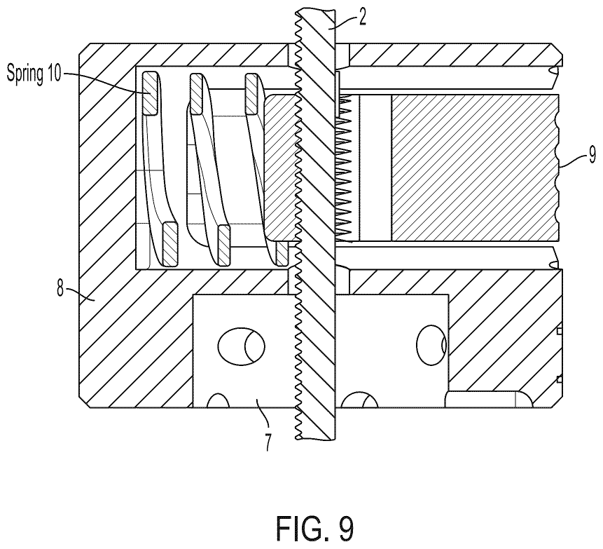

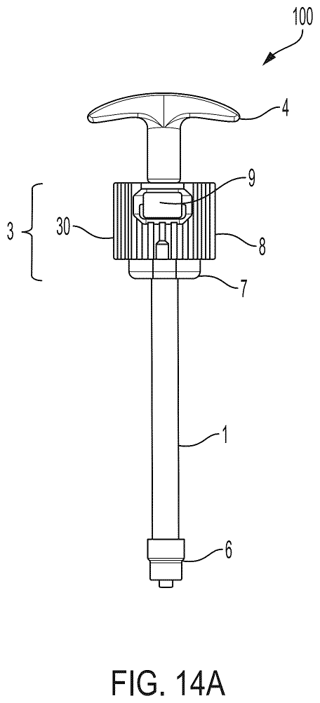

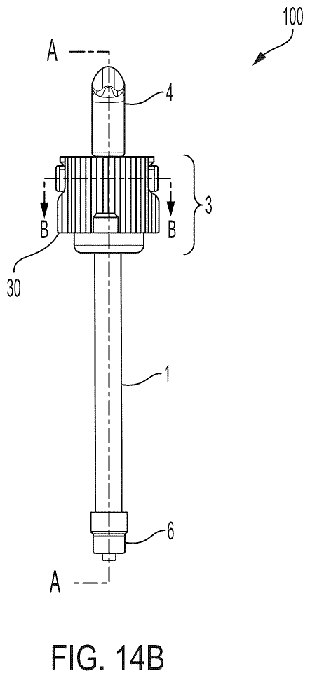

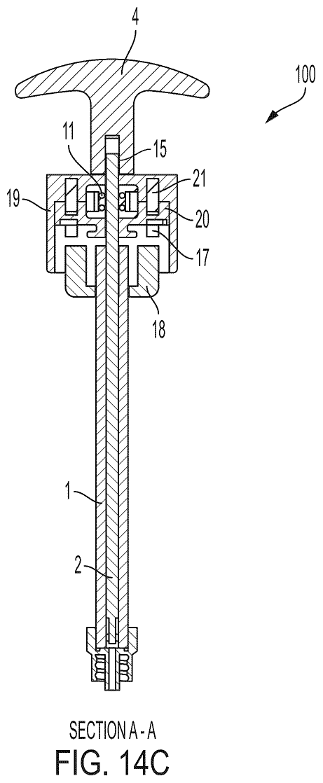

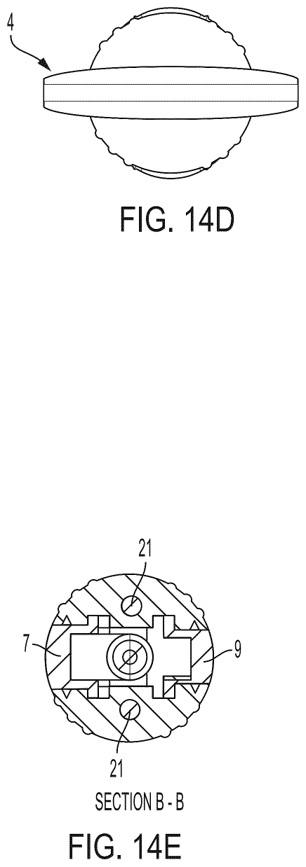

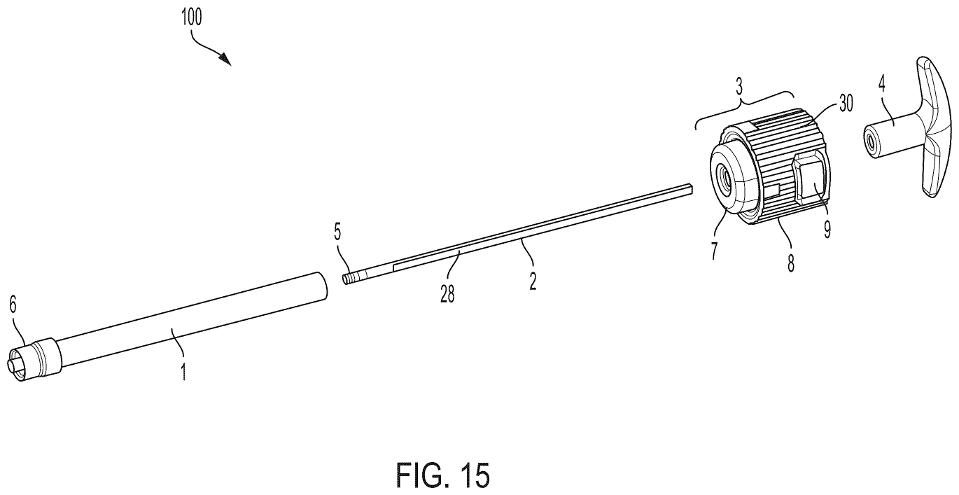

FIGS. 2A-25C illustrate examples of various aspects of threaded plunger syringes in accordance with some embodiments disclosed herein. FIGS. 2A-13C illustrate a first exemplary threaded plunger syringe, whereas FIGS. 14A-25C illustrates a second exemplary threaded syringe. The disclosure is not intended to be limited to these two exemplary threaded syringes shown, but is to be accorded the widest scope consistent with the principles and features disclosed herein. In addition, any and/or all of the features of the first and second exemplary threaded syringes can be combined in any suitable manner having combinations of all or some of the aspects described herein.

FIGS. 2A-2B, 14A-14C, 15-17, 20A-21, and 24A-C disclose various embodiments and views of syringes 100. Syringes 100 can include syringe body 1. The syringe body (or barrel) can be hollow in order to contain fluid to be dispersed via the syringe. In some embodiments, the syringe body can have a longitudinal axis extending the length of the syringe body. In some embodiments, the syringe body can have a proximal end and a distal end. In some embodiments, the distal end of the syringe body can have syringe tip 6. In some embodiments, the syringe tip can have a Luer Lock connector. The Luer Lock connector can be used to interface with an industry standard Luer Lock connection to off-the-shelf needles, Luer Lock caps, and/or cannulas.

In some embodiments, the Luer Lock connector can be used to connect various cannulas to the syringe tip to be used for a wide variety of procedures (e.g., neurological procedures) or uses. In some embodiments, the Luer Lock connector can be a male or female Luer Lock Connector. In some embodiments, the Luer Lock connector can comply with the ISO 80369-7 Luer Lock standard. In some embodiments, the Luer Lock Connector is a male Luer Lock Connector that complies with the ISO 80369-7 Luer Lock standard.

In some embodiments, the syringe can be used to deliver cells. Thus, in some embodiments, the fluid in the syringe can be a cell suspension. In some embodiments, the cells comprise stem cells, such as pluripotent stem cells (PSCs). In some embodiments, the cells comprise cells that have been differentiated from PSCs. In some embodiments, the differentiated cells comprise neurally differentiated cells. In some embodiments, the differentiated cells comprise neuronally differentiated cells. In some embodiments, the differentiated cells comprise floor plate midbrain progenitor cells, determined dopamine (DA) neuron progenitor cells, and/or dopaminergic (DA) neurons. In some embodiments, the differentiated cells comprise floor plate midbrain progenitor cells. In some embodiments, the differentiated cells comprise determined dopaminergic (DA) neuron progenitor cell. In some embodiments, the differentiated cells comprise dopaminergic (DA) neurons. Methods for differentiating PSCs to neuronally differentiated cells are known, see e.g., International PCT App. Nos. PCT/US2010/024487, PCT/JP2010/063953, PCT/US2011/037179, PCT/US2012/063339, PCT/US2021/013324, PCT/US2022/073974, each incorporated by reference in their entirety. In some embodiments, the cell suspension for delivery can include a composition of neurally differentiated stem cells, including differentiated floor plate midbrain progenitor cells, determined dopamine (DA) neuron progenitor cells, and/or dopaminergic (DA) neurons, in accord with any such methods or other methods known to a skilled artisan.

In some embodiments, the cells are engineered to correct a gene variant. In some embodiments, the gene variant is associated with a disease or condition. In some embodiments, the cells are engineered to increase expression of a gene. In some embodiments, low expression of a gene is associated with a disease or condition. In some embodiments, the cells are engineered to decrease expression of a gene. In some embodiments, high expression of a gene is associated with a disease or condition. Among the engineered cells are neurally differentiated stem cells, including differentiated floor plate midbrain progenitor cells, determined dopamine (DA) neuron progenitor cells, and/or dopaminergic (DA) neurons, that have been differentiated from PSCs. In some embodiments, the PSCs are engineered prior to the differentiation in accord with known methods, including any as described above. In some embodiments, the neutrally differentiated cells are engineered after they have been differentiated from the PSCs. Examples of neuronally differentiated cells that have been engineered include those in which a gene variant associated with Parkinson's Disease, such as GBA1, LRRK2, PRKN or SNCA, is corrected or replaced by the engineered cells. Engineered neutrally differentiated cell compositions, and methods of making the same, are known, see e.g., International PCT App. Nos. PCT/US2021/028255, PCT/US2021/028256, PCT/US2022/073967, and PCT/US2022/073973, each incorporated by reference in their entirety. In some embodiments, the cell suspension for delivery can include a composition of neurally differentiated stem cells, including differentiated floor plate midbrain progenitor cells, determined dopamine (DA) neuron progenitor cells, and/or dopaminergic (DA) neurons, that have been engineered to correct or replace a gene variant in accord with any such methods or other methods known to a skilled artisan.

In some embodiments, the cells are delivered to a target tissue. In some embodiments, the cells are delivered to a target tissue through a cannula connected to the syringe. In some embodiments, the target tissue is in a subject. In some embodiments, the target tissue is a brain tissue. In some embodiments, the target tissue is a particular region of the brain. In some embodiments, the target tissue is the striatum. In some embodiments, the target tissue is the putamen. In some embodiments, the subject or a tissue thereof has a disease or condition. In some embodiments, the subject is a human. In some embodiments, the cells treat a disease or condition.

In some embodiments, the disease or condition is a neurodegenerative disease. In some embodiments, the neurodegenerative disease is a Lewy body disease (LBD). In some embodiments, such as Parkinson's disease, Parkinson's disease dementia, or dementia with Lewy bodies (DLB). In some embodiments, the neurodegenerative disease is Parkinsonism. In some embodiments, the neurodegenerative disease is Parkinson's disease dementia. In some embodiments, the neurodegenerative disease is DLB. In some embodiments, the neurodegenerative disease is Parkinson's disease. In some embodiments, the neurodegenerative disease is idiopathic Parkinson's disease. In some embodiments, the neurodegenerative disease is a familial form of Parkinson's disease.

The syringe can be used to deliver a cell suspension (e.g., a dose of cells). In some embodiments, the size or timing of the dose(s) is determined as a function of the particular disease or condition in the subject. In some cases, the size or timing of the dose(s) for a particular disease in view of the provided description may be empirically determined. In some embodiments, the size of the dose(s) can be a predetermined amount of fluid volume dispersed from the syringe disclosed herein.

In some embodiments, the dose of cells is administered to the brain tissue of the subject. As explained above, the dose can be administered to the brain tissue from the syringe through a cannula connected to the syringe. In some embodiments, the dose of cells is administered to the striatum (e.g., putamen) of the subject. In some embodiments, the dose of cells is administered to one hemisphere of the subject's striatum (e.g., putamen). In some embodiments, the dose of cells is administered to both hemispheres of the subject's striatum (e.g., putamen).

In some embodiments, the dose of cells comprises between at or about 250,000 cells per hemisphere and at or about 20 million cells per hemisphere, between at or about 500,000 cells per hemisphere and at or about 20 million cells per hemisphere, between at or about 1 million cells per hemisphere and at or about 20 million cells per hemisphere, between at or about 5 million cells per hemisphere and at or about 20 million cells per hemisphere, between at or about 10 million cells per hemisphere and at or about 20 million cells per hemisphere, between at or about 15 million cells per hemisphere and at or about 20 million cells per hemisphere, between at or about 250,000 cells per hemisphere and at or about 15 million cells per hemisphere, between at or about 500,000 cells per hemisphere and at or about 15 million cells per hemisphere, between at or about 1 million cells per hemisphere and at or about 15 million cells per hemisphere, between at or about 5 million cells per hemisphere and at or about 15 million cells per hemisphere, between at or about 10 million cells per hemisphere and at or about 15 million cells per hemisphere, between at or about 250,000 cells per hemisphere and at or about 10 million cells per hemisphere, between at or about 500,000 cells per hemisphere and at or about 10 million cells per hemisphere, between at or about 1 million cells per hemisphere and at or about 10 million cells per hemisphere, between at or about 5 million cells per hemisphere and at or about 10 million cells per hemisphere, between at or about 250,000 cells per hemisphere and at or about 5 million cells per hemisphere, between at or about 500,000 cells per hemisphere and at or about 5 million cells per hemisphere, between at or about 1 million cells per hemisphere and at or about 5 million cells per hemisphere, between at or about 250,000 cells per hemisphere and at or about 1 million cells per hemisphere, between at or about 500,000 cells per hemisphere and at or about 1 million cells per hemisphere, or between at or about 250,000 cells per hemisphere and at or about 500,00 cells per hemisphere.

In some embodiments, the dose of cells is between at or about 1 million cells per hemisphere and at or about 30 million cells per hemisphere. In some embodiments, the dose of cells is between at or about 5 million cells per hemisphere and at or about 20 million cells per hemisphere. In some embodiments, the dose of cells is between at or about 10 million cells per hemisphere and at or about 15 million cells per hemisphere.

In some embodiments, the dose of cells is between about 3×10⁶ cells/hemisphere and 15×10⁶ cells/hemisphere. In some embodiments, the dose of cells is about 3×10⁶ cells/hemisphere. In some embodiments, the dose of cells is about 4×10⁶ cells/hemisphere. In some embodiments, the dose of cells is about 5×10⁶ cells/hemisphere. In some embodiments, the dose of cells is about 6×10⁶ cells/hemisphere. In some embodiments, the dose of cells is about 7×10⁶ cells/hemisphere. In some embodiments, the dose of cells is about 8×10⁶ cells/hemisphere. In some embodiments, the dose of cells is about 9×10⁶ cells/hemisphere. In some embodiments, the dose of cells is about 10×10⁶ cells/hemisphere. In some embodiments, the dose of cells is about 11×10⁶ cells/hemisphere. In some embodiments, the dose of cells is about 12×10⁶ cells/hemisphere. In some embodiments, the dose of cells is about 13×10⁶ cells/hemisphere. In some embodiments, the dose of cells is about 14×10⁶ cells/hemisphere. In some embodiments, the dose of cells is about 15×10⁶ cells/hemisphere.

In some embodiments, the dose of cells is about 5×10⁶ cells in each putamen. In some embodiments, the dose of cells is about 10×10⁶ cells in each putamen.

In some embodiments, the number of cells administered to the subject is between about 0.25×10⁶ total cells and about 20×10⁶ total cells, between about 0.25×10⁶ total cells and about 15×10⁶ total cells, between about 0.25×10⁶ total cells and about 10×10⁶ total cells, between about 0.25×10⁶ total cells and about 5×10⁶ total cells, between about 0.25×10⁶ total cells and about 1×10⁶ total cells, between about 0.25×10⁶ total cells and about 0.75×10⁶ total cells, between about 0.25×10⁶ total cells and about 0.5×10⁶ total cells, between about 0.5×10⁶ total cells and about 20×10⁶ total cells, between about 0.5×10⁶ total cells and about 15×10⁶ total cells, between about 0.5×10⁶ total cells and about 10×10⁶ total cells, between about 0.5×10⁶ total cells and about 5×10⁶ total cells, between about 0.5×10⁶ total cells and about 1×10⁶ total cells, between about 0.5×10⁶ total cells and about 0.75×10⁶ total cells, between about 0.75×10⁶ total cells and about 20×10⁶ total cells, between about 0.75×10⁶ total cells and about 15×10⁶ total cells, between about 0.75×10⁶ total cells and about 10×10⁶ total cells, between about 0.75×10⁶ total cells and about 5×10⁶ total cells, between about 0.75×10⁶ total cells and about 1×10⁶ total cells, between about 1×10⁶ total cells and about 20×10⁶ total cells, between about 1×10⁶ total cells and about 15×10⁶ total cells, between about 1×10⁶ total cells and about 10×10⁶ total cells, between about 1×10⁶ total cells and about 5×10⁶ total cells, between about 5×10⁶ total cells and about 20×10⁶ total cells, between about 5×10⁶ total cells and about 15×10⁶ total cells, between about 5×10⁶ total cells and about 10×10⁶ total cells, between about 10×10⁶ total cells and about 20×10⁶ total cells, between about 10×10⁶ total cells and about 15×10⁶ total cells, or between about 15×10⁶ total cells and about 20×10⁶ total cells.

In certain embodiments, the cells, or individual populations of sub-types of cells, are administered to the subject at a range of about 5 million cells per hemisphere to about 20 million cells per hemisphere or any value in between these ranges. Dosages may vary depending on attributes particular to the disease or disorder and/or patient and/or other treatments. In some embodiments, the patient is administered multiple doses, and each of the doses or the total dose can be within any of the foregoing values. In some embodiments, the dose of cells comprises the administration of from or from about 5 million cells per hemisphere to about 20 million cells per hemisphere, each inclusive. In some embodiments, the dose of cells, e.g., overexpressing cells, is administered to the subject as a single dose or is administered only one time within a period of two weeks, one month, three months, six months, 1 year or more.

In the context of cell transplant, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as a day. Thus, in some contexts, the dose is a single or continuous administration of the specified number of cells, given or initiated at a single point in time. In some contexts, however, the dose is administered in multiple injections or infusions in a single period, such as by multiple infusions over a single day period.

Thus, in some aspects, the cells of the dose are administered in a single pharmaceutical composition. In some embodiments, the cells of the dose are administered in a plurality of compositions, collectively containing the cells of the dose.

The working volume of the syringe body can be any working volume. In some embodiments, the syringe body can have a working volume of at least about 10 microliters, at least about 50 microliters, at least about 250 microliters, at least about 450 micro liters, at least about 500 microliters, at least about 550 microliters, at least about 750 microliters, or at least about 1000 microliters. In some embodiments the syringe body can have a working volume of at most about 10 mL, at most about 5 mL, at most about 2 mL, at most about 1 mL, at most about 750 microliters, at most about 550 microliters, at most about 500 microliters, at most about 450 microliters, at most about 250 microliters, at most about 100 microliters, or at most about 50 microliters. In some embodiments, the working volume of the syringe body can be about 10-1000 microliters, about 250-750 microliters, about 450-550 microliters, or about 500 microliters.

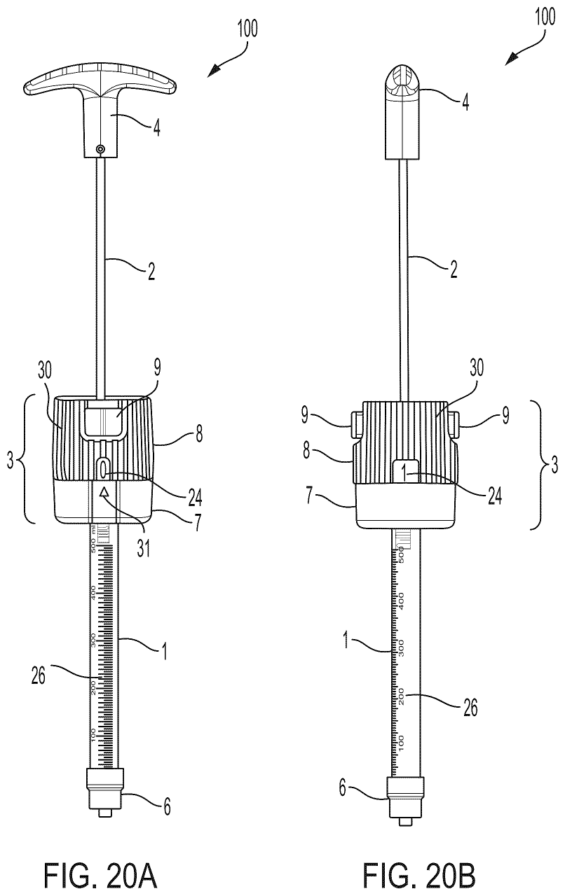

In some embodiments, the syringe body can include gradations at predetermined volume increments as shown by gradations 26 in FIGS. 2A, 20A-B, and 24A. In some embodiments, the syringe body can have major gradations at every 10, 50, 100, or 500 microliter increment. In some embodiments, the syringe body can have minor gradations at every 5, 10, 50 or 100 microliter increment. In some embodiments, the syringe body can have sub-minor gradations at every 1, 5, 10, or 50 microliter increment. In some embodiments, the gradations can be markings on the syringe body. In some embodiments, the markings can be black or dark color markings on the syringe body.

In some embodiments, the syringe body can be manufactured of materials that are biocompatible (e.g., silicone that is USP Class VI), inert, DMSO compatible, shatter resistant, and/or compatible with use in an MRI scanner (non-magnetic). In some embodiments, the syringe body can be manufactured of borosilicate glass or cyclic olefin polymer (COP). In some embodiments, the syringe body can be transparent. In some embodiments, the syringe body can have transparency similar to borosilicate glass or clear polycarbonate.

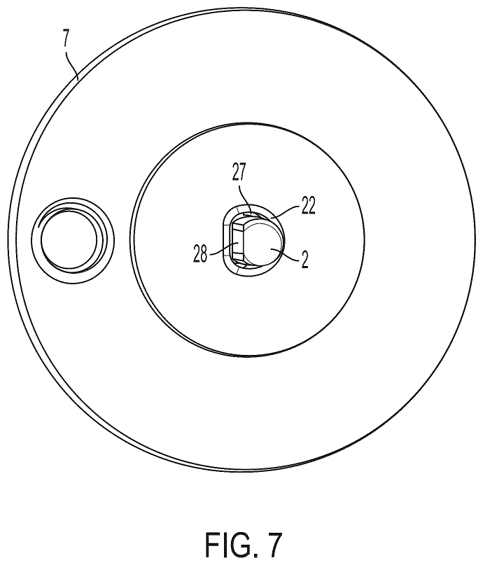

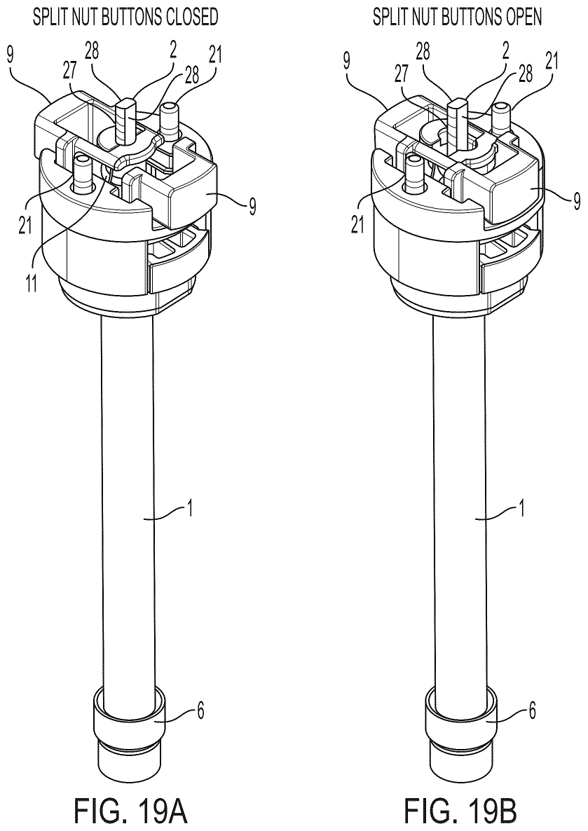

The syringes disclosed herein can also include threaded plunger 2. In some embodiments, the threaded plunger can be a syringe plunger with a threaded design that allows for extremely precise plunger movement. In some embodiments, a portion of the threaded plunger can be within the syringe body. In some embodiments, the threaded plunger can be configured to move longitudinally in the syringe body. In other words, the threaded plunger can move in either direction of the longitudinal axis of the syringe body. The threaded plunger can include threads. In some embodiments, the threaded plunger can include at least one longitudinal unthreaded portion that runs along the longitudinal axis of the threaded plunger. In some embodiments, the at least one longitudinal unthreaded portion can be a flat portion of the threaded plunger that runs along the longitudinal axis of the threaded plunger. In other words, the longitudinal unthreaded flat portion can be as if a chord of the threaded plunger's cross-sectional circle was cut longitudinally down the threaded plunger's longitudinal axis. An example of a flat longitudinal unthreaded portion 28 of threaded plunger 2 is shown in FIG. 7. As illustrated in FIG. 7, the longitudinal flat unthreaded portion gives the threaded plunger a “D” shaped cross section. In some embodiments, the threaded plunger can include more than one longitudinal unthreaded portion. For example, FIGS. 19A-19B illustrate threaded plunger 2 with two flat longitudinal unthreaded portions 28 on each side of the threaded portion 27 of the threaded plunger. In some embodiments, the threaded plunger is a left-handed threaded plunger.

In some embodiments, the threaded plunger can include plunger tip 5 on a distal end of the threaded plunger. In some embodiments, the plunger tip can be a resilient fluid tight member. In some embodiments, the plunger tip can be at the distal end of the threaded plunger that is in the fluid path of the syringe. In some embodiments, the plunger tip is configured to keep fluid away from the proximal end of the threaded plunger. As such, when the threaded plunger moves longitudinally in the syringe body towards the distal end of the syringe body, any fluid in the syringe body can be pushed away from the plunger tip in the longitudinal direction of the syringe body toward the distal end.

Applicant discovered that when the plunger tip (e.g., a plastic material such as ETFE or PTFE) is in the syringe body and exposed to elevated temperatures or other stresses such as during sterilization, the plunger tip can deform (e.g., creep or cold flow) as it tries to expand in the syringe body which can prevent such expansion (e.g., non-expandable or minimally expandable syringe body such as glass). This deformity can cause the plunger tip to no longer seal properly or keep fluid away from the proximal end of the threaded plunger while in the syringe body. In other words, the syringe would fail a leak-when-loaded test. To combat this, Applicant created a new syringe tip.

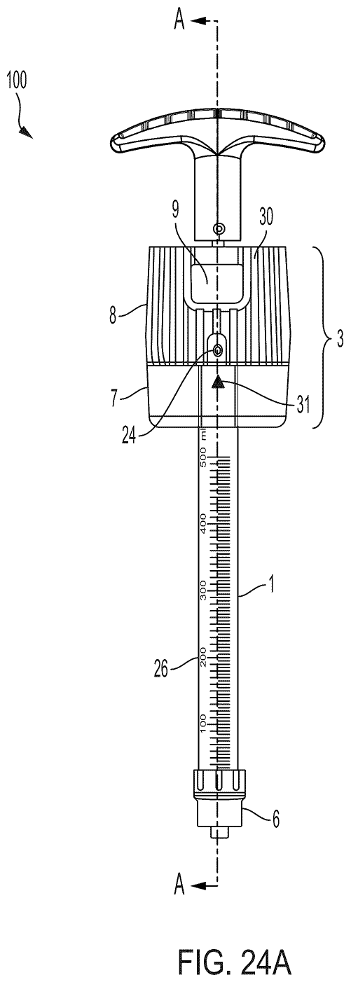

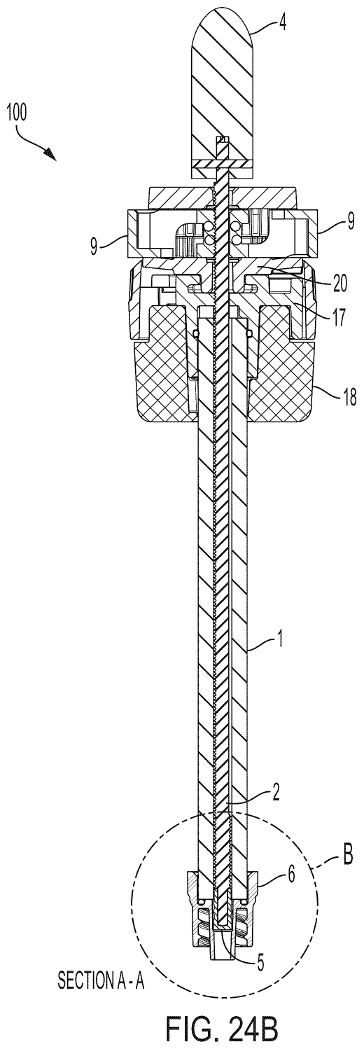

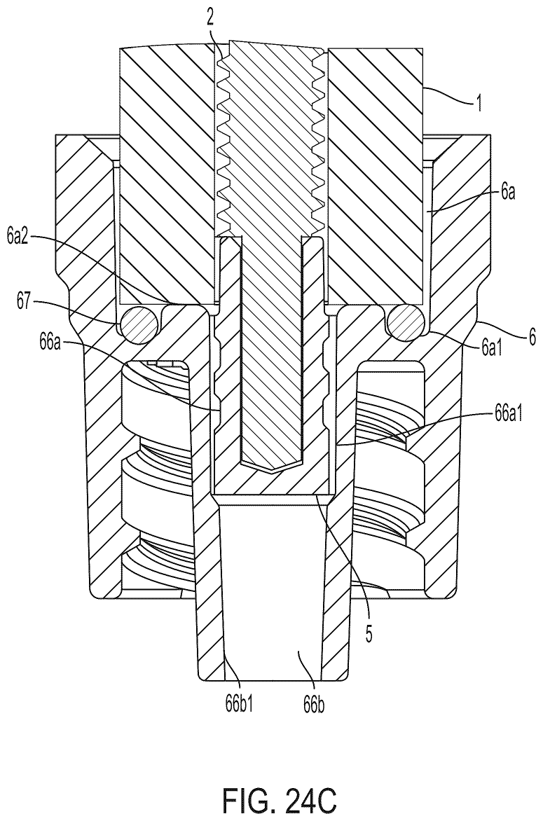

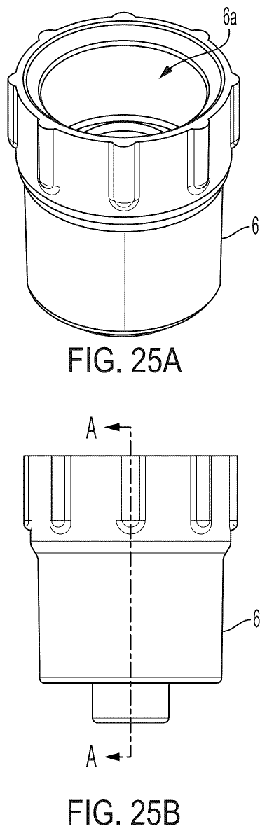

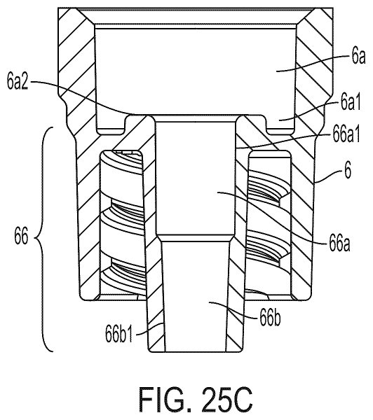

FIGS. 25A-C illustrate various views of syringe tip 6. As shown in cross section FIG. 25C, the syringe tip can include receptacle or recess 6a. In some embodiments, the receptacle can be configured to receive and/or support the syringe body. For example, FIG. 24C illustrates an enlarged view of a portion of syringe body 1 within receptacle 6a. In some embodiments, the receptacle can be a shape (e.g., cylindrical) configured to receive a syringe body. In some embodiments, the receptacle can include a shoulder 6a2 configured to support the syringe body. In some embodiments, the receptacle can include an indent 6a1. In some embodiments, the indent can be configured to receive and/or house an O-ring such as O-ring 67 as shown in FIG. 24C.

In some embodiments, the syringe tip can include a channel 66. The channel can be fluidly connected to the inside of the syringe body such that any fluid inside of the syringe can exit the syringe tip through the channel. In some embodiments, the channel can include a first proximal portion 66a and a second distal portion 66b. In some embodiments, the first portion of the channel can configured to receive at least a portion of the plunger tip. For example, in some embodiments, a portion of the plunger tip can be stored in the first portion of the channel whenever the syringe is exposed to elevated temperatures or other stresses (e.g., during sterilization). FIG. 24C illustrates plunger tip 5 (and a portion of plunger 2) within first portion 66a of the channel of the syringe tip. As shown in FIG. 24C, the plunger tip may not be compressed or pushed up against the surface 66a1 of the first portion 66a (i.e., there can be a gap or space between the plunger tip and the interior surface of the first portion of the channel). In other words, the first portion of the channel can allow at least a portion of the plunger tip to not be compressed radially (unlike when the plunger tip is within the syringe body). This can allow the plunger tip to expand and/or contract (during sterilization) without experiencing any additional stress (such as that when it is compressed within a syringe body). In some embodiments, the plunger tip may only be in the first portion of the channel of the syringe tip when the syringe is unloaded such as during sterilization, shipping, storage, or whenever it may experience elevated temperatures or additional stresses. Before loading the syringe, the plunger tip would be retracted within the syringe body and the seal between the syringe body and plunger tip can be maintained.

In some embodiments, the first portion of the channel may be a shape configured to receive at least a portion of the plunger tip. In some embodiments, the shape can be cylindrical or frustroconical. For example, in some embodiments, the interior surface (66a1) of the first portion of the channel may be a tapered surface such that it forms a frustroconical shape that tapers towards the distal end of the syringe.

In some embodiments, the second portion of the channel may be a shape similar to that of the first portion. In some embodiments, the shape can be cylindrical or frustroconical. For example, in some embodiments, the interior surface (66b1) of the second portion of the channel may be a tapered surface such that it forms a frustroconical shape that tapers towards the distal end of the syringe. In some embodiments, the second portion of the channel can be narrower than the first portion of the channel (e.g., have all diameters within the second portion smaller than all diameters within the first portion of than channel). This can be in order to prevent the plunger tip from entering into the second portion of the channel as shown in FIG. 24C.

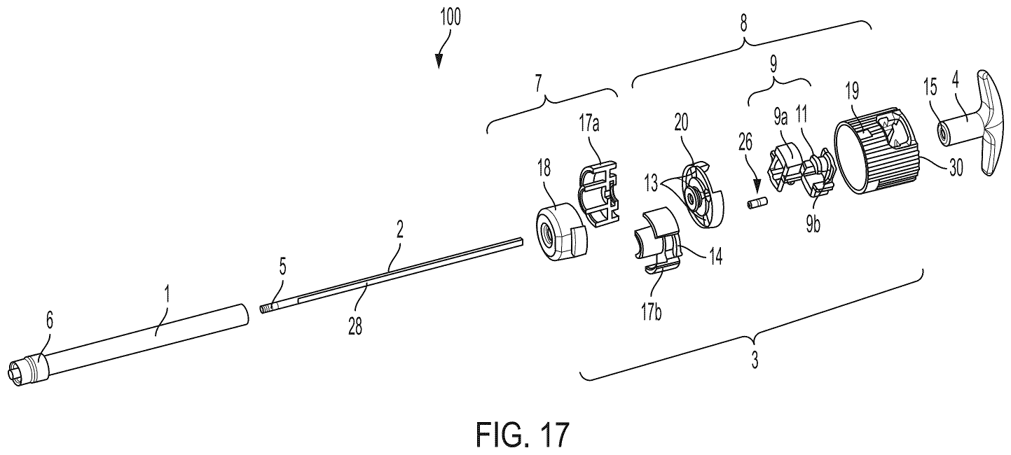

In some embodiments, threaded plunger 2 can include plunger handle 4 on a proximal end of the threaded plunger. In some embodiments, the plunger handle can allow an operator to manually draw fluid into the syringe body and/or prime the syringe. In some embodiments, the plunger handle can be attached to the threaded plunger via plunger handle retainer 15. In some embodiments, the plunger handle retaining mechanism can be any retaining mechanism such as adhesives, Velcro, screws, threaded inserts, and variety of others. In some embodiments, the plunger handle retaining mechanism can be a threaded insert in the plunger handle such as plunger handle retainer 15 shown in at least FIGS. 14C and 17. In some embodiments, the plunger handle retainer can be a set screw such as plunger handle retainer 15 shown in at least FIGS. 2A-2B.

The plunger handle can come in a variety of shapes and sizes. In some embodiments, the plunger handle can be cylindrical, square, spherical, rectangular, trapezoidal, a variety of other polygons. In some embodiments, the plunger handle can be a pull tab or a T-grip as that shown in at least FIG. 17. As discussed in more detail below, the purpose of the plunger handle can be to allow for the threaded plunger to move in the longitudinal direction (without any rotation of any component of the syringe) within the syringe body when the plunger disengagement mechanism is activated. In some embodiments, the plunger handle does not rotate while the threaded plunger moves longitudinally in the syringe body. As such, the plunger handle may not be rotatable either.

The syringes disclosed herein can also include syringe body mount 3. In some embodiments, the syringe body mount can be connected to the proximal end of the syringe body. In some embodiments, a portion of the syringe body mount can be fixed to the proximal end of the syringe body. The portion of the syringe body mount fixed to the proximal end of the syringe body can be non-movable with respect to the syringe body.

In some embodiments, when the syringe body mount is in a first configuration, the syringe body mount can allow the syringe to operate as a threaded plunger syringe for precisely controlled fluid delivery. In some embodiments, when the syringe body mount is in a second configuration, the syringe body mount can allow the syringe to operate as a conventional plunger syringe. For example, in the second configuration, the syringe body can be loaded with fluid and/or primed by pulling the plunger handle towards the proximal end of the syringe such that fluid is sucked into the syringe body. Likewise, in the second configuration, fluid can be dispersed from the syringe body by pushing the plunger handle toward the distal end of the syringe.

In some embodiments, the threaded plunger can pass through the syringe body mount and into the syringe body. In some embodiments, a portion of the syringe body mount can be configured to prevent the threaded plunger from rotating within the syringe. When the syringe body mount is in the first configuration, rotation of a portion of the syringe body mount (instead of the plunger handle) can cause the threaded plunger to move longitudinally in the syringe body. In some embodiments, the portion of the syringe body mount can be manually rotated to enable continuous delivery of a fluid in the syringe. In some embodiments, when the syringe body mount is in the first configuration, clockwise rotation of a portion of the syringe body mount can cause the threaded plunger to move longitudinally towards the distal end of the syringe body and counterclockwise rotation of the of a portion of the syringe body mount can cause the threaded plunger to move longitudinally towards the proximal end of the syringe body.

When the syringe body mount is in the first configuration, a portion of the syringe body mount can engage with the threads of the threaded plunger. As such, the portion of the syringe body that engages with the threads of the threaded plunger can include threads that are complimentary to and/or configured to mate with the threads of the threaded plunger.

In some embodiments, syringe body mount 3 can include control base 7 and control knob 8. In some embodiments, the control knob can be a control wheel. In some embodiments, the control wheel can be a rotating wheel. In some embodiments, the control knob can be used to dispense fluid from the syringe body by turning it clockwise. In some embodiments, the torque to rotate the control knob from one position to the next may be at most about 72.5 Ncm. As described further herein, rotation of the control knob can cause longitudinal axial translation of the threaded plunger. In some embodiments, the control base can be an enclosure fixed to the syringe body. As described further herein, the control base and/or control knob can display visual feedback.

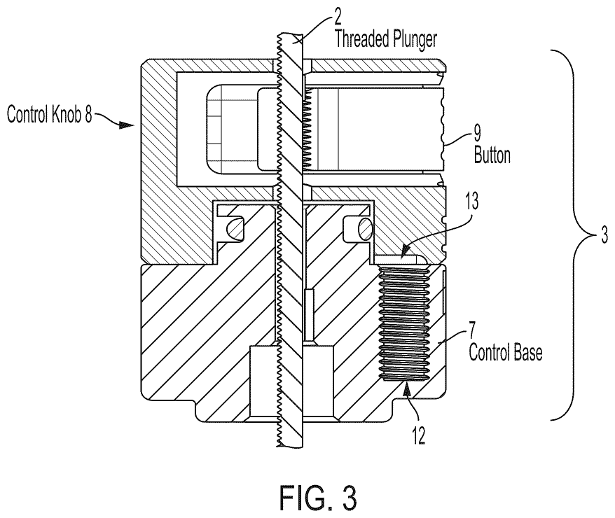

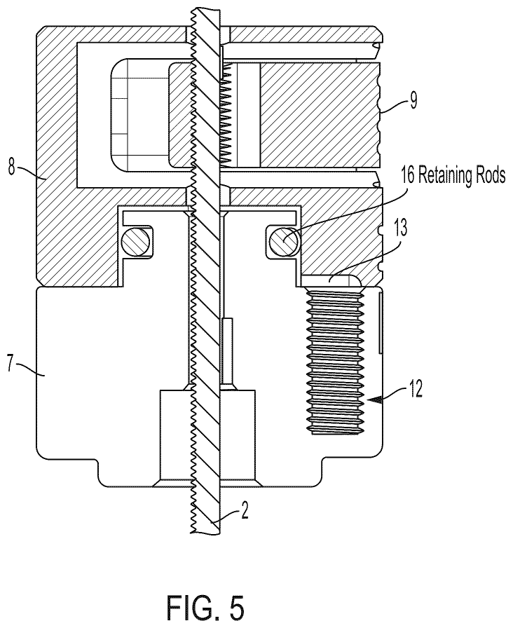

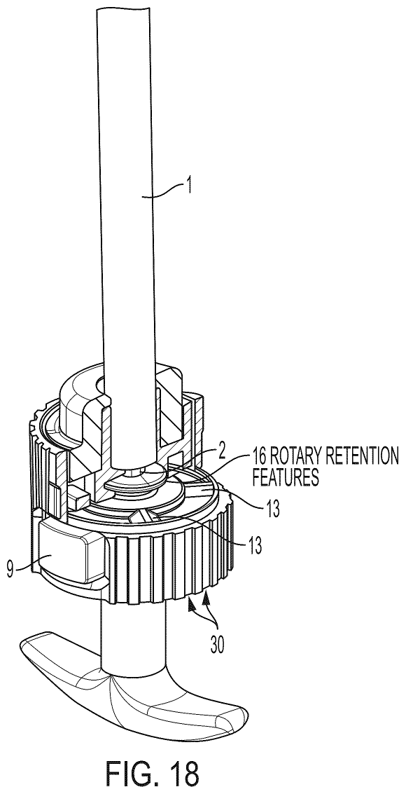

As shown in FIG. 3, the threaded plunger can pass through the control knob, the control base, and into the syringe body. In some embodiments, the control base can be fixed to the proximal end of the syringe body such that the control base does not move with respect to the syringe body. In some embodiments, control knob can be connected to the control base such that the control knob is moveable with respect to the control base. For example, FIG. 5 illustrates control knob 8 and control base 7 moveably fixed together via retainer(s) 16 (e.g., pins, retaining rods, etc.). In addition, FIG. 18 illustrates retainer 16, which is a rotary retainer that can allow for the control knobs rotation with respect to the control base. In some embodiments, the control base can be configured to prevent the threaded plunger from rotating within the syringe. In some embodiments, the control base can include an aperture with the shape of the cross section of the threaded plunger such that when the threaded plunger passes through the aperture in the control base, the threaded plunger cannot rotate. FIG. 7 illustrates control base 7 with “D” shaped aperture 22 to accommodate the threaded plunger with a “D” shaped cross section.

In some embodiments, a portion of the control knob can engage with the threads of the threaded plunger. In other words, in the first configuration (which can be the default configuration) a portion of the control knob can engage (or rest against) the threads of the threaded plunger. In some embodiments, the control knob can be configured to engage with threads of the threaded plunger such that rotation of the control knob with respect to the control base can cause the threaded plunger to move longitudinally in the syringe body.

In some embodiments, the control knob can be moveably fixed together with the control base to ensure that progression of the complimentary threads of the control knob move the threaded plunger in the longitudinal direction in the syringe. In some embodiments, the control knob can be rotatably connected to the control base such that the control knob rotates with respect to the control base (and thus also with respect to the syringe body). In some embodiments, the control knob can be rotated to progress the complimentary threads of the control knob along the threads of the threaded plunger. In some embodiments, the control knob is configured to engage with the threads of the threaded plunger such that clockwise rotation of the control knob with respect to the control base (and thus the syringe body) causes the threaded plunger to move longitudinally towards the distal end of the syringe body and counterclockwise rotation of the control knob with respect to the control base causes the threaded plunger to move longitudinally towards the proximal end of the syringe body. In some embodiments, the control knob can manually be rotated (instead of the plunger handle) to continuously deliver fluid from the syringe while the control base stays in place.

When the syringe body mount is in the second configuration, the threaded plunger can move longitudinally in the syringe body without rotation of the syringe body mount (or, in some embodiments, rotation of any other component of the syringe). In some embodiments, when the syringe body mount is in the second configuration, the threaded plunger can move longitudinally in the syringe body by moving the threaded plunger longitudinally (e.g., by pulling or pushing the threaded plunger or plunger handle of the threaded plunger).

When the syringe body mount is in the second configuration, a portion of the syringe body mount may not be engaged with the threads of the threaded plunger. As stated above, in some embodiments, the threaded plunger does not rotate whenever it moves longitudinally in the syringe body. In some embodiments, the threaded plunger never rotates in the syringe.

In some embodiments, the syringe body mount can include plunger disengagement mechanism configured to disengage the syringe body mount from the threads of the threaded plunger. When the plunger disengagement mechanism is activated, the syringe body mount can be in its second configuration. In some embodiments, the plunger disengagement mechanism can include at least one depressible button 9. As described further herein, the at least one depressible button can be a button on the control knob that the user presses to disengage the thread of the threaded plunger to allow for manual longitudinal axial movement of the threaded plunger. In some embodiments, the activation force of the at least one depressible button can be no more than about 10 N.

In some embodiments, when the at least one depressible button is depressed, the syringe body mount can enter its second configuration (i.e., the syringe body mount disengages from the threads of the threaded plunger). In some embodiments, when more than one depressible button are depressed (e.g., simultaneously), the syringe body mount can enter its second configuration. In some embodiments, depressing the at least one depressible button can allow for temporary release of the threaded plunger from the threaded mechanism of the syringe body mount and/or control knob of the syringe body mount while the at least one depressible button is depressed. While the at least one depressible button is depressed (i.e., the second configuration), the threaded plunger can move in both directions of the longitudinal axis of the syringe body without its threads being engaged by any component of the syringe (besides possibly the plunger handle).

In some embodiments, the control knob can include the plunger disengagement mechanism. In some embodiments, the plunger disengagement mechanism and/or syringe body mount can include at least one spring and/or at least one 0-ring to keep the syringe body mount in the first configuration by default. This plunger disengagement mechanism can disengage the control knob from the threads of the threaded plunger such that the threaded plunger can be configured to move longitudinally in the syringe body without rotation of the control knob. In some embodiments, the threaded plunger can engage the complimentary threads of the at least one depressible button in the first configuration. In some embodiments, the control knob can include the at least one depressible button. In some embodiments, the at least one depressible button can be moveably fixed to the control knob. In some embodiments, the threaded plunger can pass through the control knob, the at least one depressible button of the control knob, the control base, and into the syringe body. In some embodiments, the at least one depressible button is flush with, or slightly recessed from, the surface of the syringe body mount and/or control knob of the syringe body mount. In some embodiments, the at least one depressible button can have enough surface area to allow an operator's finger to comfortably depress the at least one depressible button.

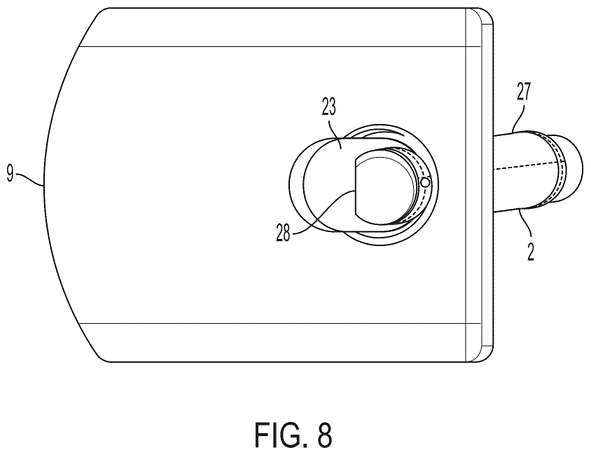

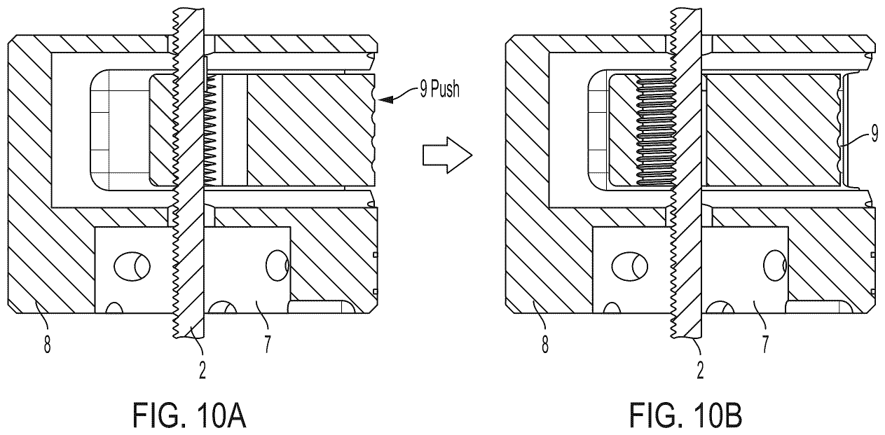

In the first configuration, the control knob can be rotated to progress the threads in the at least one depressible button along the threads of the threaded plunger. In some embodiments, to allow the threaded plunger to be disengaged from the syringe body mount, control knob, and/or at least one depressible button of the control knob, the at least one depressible button can include slot 23 with only one side being threaded as shown in FIG. 8. In some embodiments, the first configuration (i.e., default configuration) with the threads of threaded plunger being engaged with the control knob can be accomplished using spring 10 in the control knob. The spring can be configured to cause (e.g., push the threaded portion of the at least one depressible button against the threads of the threaded plunger) the at least one depressible button to engage with the threads of the threaded plunger and to disengage with the threads of the threaded plunger when the at least one depressible button is depressed. In some embodiments, to disengage the threaded plunger from the syringe body mount, control knob, and/or at least one depressible button of the control knob, the at least one depressible button is depressed or compressed against the spring to move the threaded plunger to the unthreaded side of the slot, as shown in FIGS. 10A-10B, for example. In some embodiments, the spring can provide enough resistance to prevent an accidental depression of the at least one depressible button. In some embodiments, the spring can provide resistance such that the at least one depressible button is not too difficult for an operator to depress.

In some embodiments, the threaded plunger can engage the threads of more than one depressible button in the first configuration. In some embodiments, the more than one depressible buttons (e.g., 9a and 9b in FIG. 17) can be on opposite sides of the syringe body mount and/or control knob of the syringe body mount. In some embodiments, the control knob can include more than one compressible button. In some embodiments, the at least one button can made of plastic (e.g., HMWPE or PTFE) or brass. In some embodiments, the more than one compressible buttons can be moveably fixed to the control knob. In the first configuration, the control knob can be rotated to progress the threads in the more than one depressible button along the threads of the threaded plunger. In some embodiments, the first configuration (i.e., default configuration) with the threads of threaded plunger being engaged with the control knob can be accomplished using at least one O-ring 11 in the control knob. In some embodiments, the at least one O-ring can cause (e.g., pull the threads of the more than one depressible buttons against the threads of the threaded plunger) the more than one depressible buttons to engage with the threads of the threaded plunger and to disengage with the threads of the threaded plunger when the more than one depressible buttons are depressed. In some embodiments, to disengage the threaded plunger from the syringe body mount, control knob, and/or more than one depressible buttons of the control knob, the more than one depressible buttons are depressed or compressed forcing the at least one O-ring to stretch and move the threads of the more than one depressible buttons away from the threaded plunger, as shown in FIGS. 19A-19B for example. In some embodiments, the at least one O-ring can provide enough resistance to prevent an accidental depression of the more than one depressible buttons. In some embodiments, the at least one O-ring can provide resistance such that the more than one depressible buttons are not too difficult for an operator to depress.

In some embodiments, the syringe body mount can be configured to provide tactile feedback to an operator of the syringe when a predetermined amount of fluid volume is dispersed from the syringe. As described above, it was difficult for an operator of the Hamilton Company syringe to know how much fluid was being dispersed from the syringe as the operator rotated the plunger handle. As such, the syringe body mounts described herein can include a tactile feedback mechanism. The tactile feedback mechanism can be a mechanism that provides tactile feedback such as a vibration or physical click for each predetermined amount of fluid volume dispersed. In some embodiments, the control knob and/or the control base can include the tactile feedback mechanism.

In some embodiments, the syringe body mount can include a resistance member and a plurality of detents such that a portion of the syringe body mount can rotate until the resistance member reaches a detent that can temporarily prevent rotation of the portion of the syringe body mount. The tactile feedback can be provided to the operator when a resistance member reaches a detent. After reaching a first detent, the operator can then further rotate at least part of the syringe body mount. The portion of the syringe body mount can continue to rotate until the resistance member reaches a second detent. The distance between the plurality of detents can correspond to a predetermined amount of fluid volume. In some embodiments, the distance between the plurality of detents can be the rotational distance or arc distance of rotation of the portion of the syringe body mount for the resistance member to move from one detent to another. Thus, when an operator rotates a portion of the syringe body mount such that the resistance member moves from one detent to another, this can signal that a specific predetermined amount of fluid has been dispersed from the syringe.

In some embodiments, the control base or the control knob can include the resistance member(s). In some embodiments, the control base or the control knob can include the plurality of detents. In some embodiments, if the control base includes the resistance member, the control knob can include the plurality of detents and vice versa.

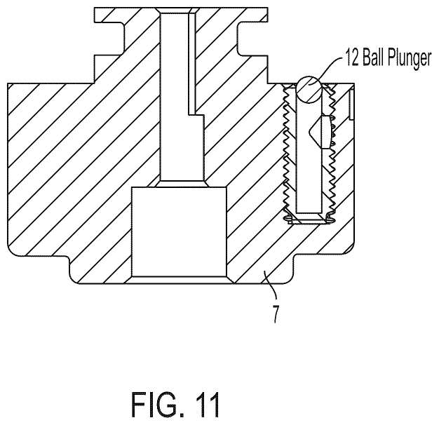

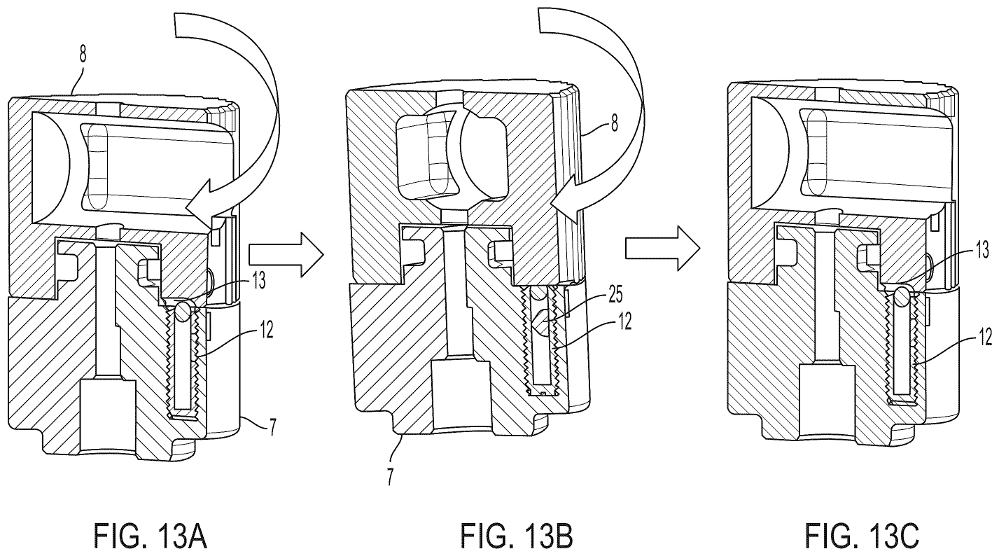

In some embodiments, the resistance member can be any element that provides resistance as a portion of the syringe body mount rotates. For example, in some embodiments, the resistance element can be a ball plunger or a flexure. FIGS. 11-13 illustrate one example of a tactile feedback mechanism utilizing ball plunger 12. Although FIGS. 11-13 illustrate ball plunger 12 in the control base and plurality of detents 13 in control knob, these can be switched such that the ball plunger is in the control knob and the plurality of detents are in the control base. In some embodiments, the ball plunger can be threaded into control base and/or control knob. In some embodiments, the ball plunger can include thread locking element 25. In some embodiments, the ball plunger can match with the detents such that the ball of the ball plunger can fit into the detent. In some embodiments, the detents can be depressions and/or cutouts in a surface of the control knob or control base. Accordingly, the ball plunger can extend outward from a surface of the control knob or control base (as shown in FIG. 11), such that when a detent is lined up with the ball plunger, the ball of the ball plunger can fit in the detent. As shown in FIGS. 13A-C, as the control knob is rotated, the ball plunger can be depressed between a surface of the control knob and a surface of the control base until the ball plunger reaches the next detent, where the ball plunger can “snap” or “pop” or “click” into the detent providing tactile feedback to the user. In some embodiments, the control base can include a ball plunger and the control knob can include a plurality of detents configured to fit a portion of the ball plunger such that as the control knob rotates, the ball plunger can be depressed between the control base and control knob until the ball plunger reaches a detent where the ball plunger fits into the detent providing tactile feedback to the operator. The process can be repeated for each detent. In some embodiments, the tactile feedback mechanism can also provide audio feedback to the operator of the syringe. For example, the ball plunger snapping into a detent can also make a click or popping sound.

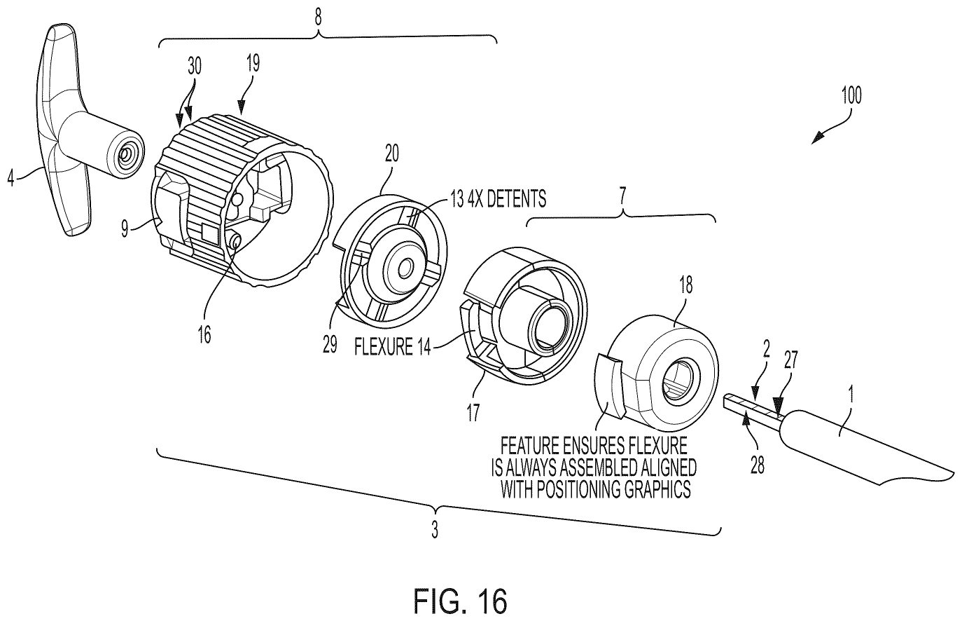

FIG. 16 illustrates another example of a tactile feedback mechanism utilizing flexure 14. Although FIGS. 16-17 illustrate flexure 14 on a surface of the control base and plurality of detents 13 on a surface of the control knob, these can be switched such that the flexure is in the control knob and the plurality of detents are in the control base. In some embodiments, a control base or control knob can include flexure holder 17. In some embodiments, the flexure holder can be two part flexure holder, wherein one part 17b has the flexure and the second part 17a does not. In some embodiments, the control base can also include control base mount 18. The control base mount can be connected to the proximal end of the syringe body. In some embodiments, the control base mount can be fixed to the proximal end of the syringe body. The control base mount can be non-movable with respect to the syringe body. The control base mount can also be connected to the flexure holder. In some embodiments, the control base mount can be fixed to the flexure holder such that they are non-moveable with respect to each other.

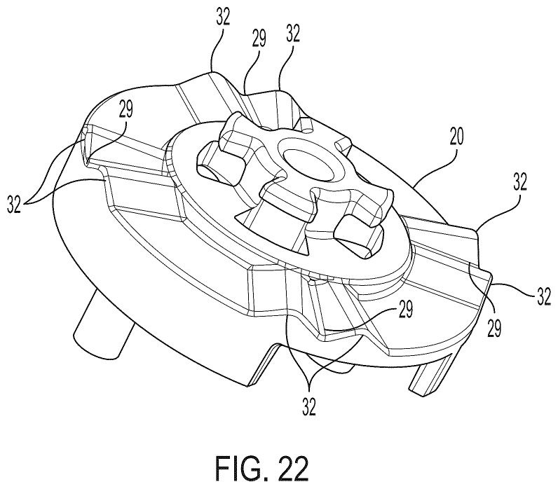

In some embodiments, the detents can be ramps, slopes, gradients, etc. on a surface of the control knob (as shown in FIG. 16) or control base. In some embodiments, a detent can include multiple hills, ramps, slopes, gradients, etc., with a valley, gap, or slot 29 between the ramps, slopes, gradients, etc. where the flexure can sit or rest. For example, FIG. 22 illustrates detent holder with four detents, wherein each detent includes two hills, ramps, slopes, gradients, etc. 32 with a valley, gap, or slot 29 between the two hills, ramps, slopes, gradients, etc. 32. Accordingly, as the control knob is rotated, the flexure on a surface of the control base (or control knob) can come into contact with a detent on a surface of the control knob (or control base if the flexure is on the control knob). This can cause the flexure to experience resistance as it is pushed against the detent until it reaches the portion of the detent where the flexure can rest or sit. For example, in some embodiments, as the control knob is rotated, the flexure can come into contact with a detent ramp of a detent that forces the flexure to bend creating resistance. Once the flexure reaches the top of the ramp, the flexure can “snap,” “pop,” or “click” into the gap, valley, or slot of the detent to rest or sit providing tactile feedback to the user.

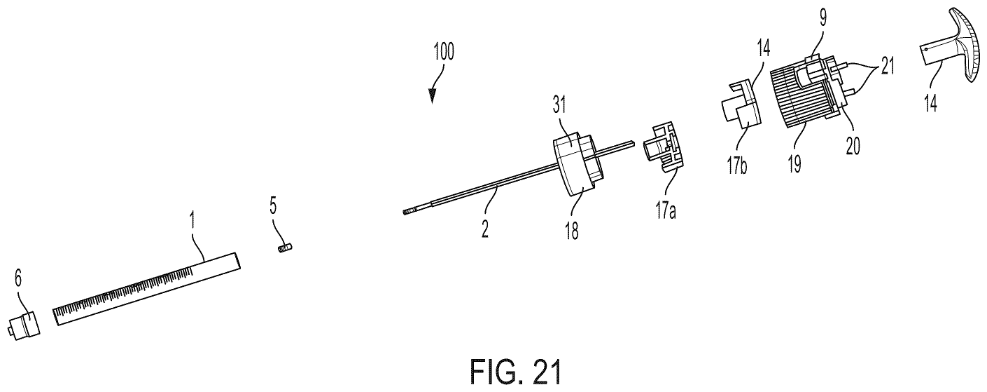

In some embodiments, the control knob can include a detent holder 20 that includes the plurality of detents on a surface of the detent holder. In some embodiments, the control knob can also include control knob casing 19 connected to the detent holder. In some embodiments, the control knob casing is fixed to the detent holder such that they are non-moveable with respect to each other. In some embodiments, the control knob casing can be connected to the detent holder via retainer 21 (e.g., pins). In some embodiments, retainer(s) 21 can be integral to the detent holder as shown in FIG. 21.

In some embodiments, the control base can include a flexure and the control knob can include a plurality of detents configured to fit a portion of the flexure such that as the control knob rotates, the flexure can experience resistance when it reaches a detent until the flexure fits into a portion of the detent providing tactile feedback to the operator. In some embodiments, the flexure can experience resistance throughout rotation until it reaches a detent. The process can be repeated for each detent. In some embodiments, the tactile feedback mechanism can also provide audio feedback to the operator of the syringe. For example, the flexure snapping into a detent can also make a click or popping sound.

In some embodiments, the control base can include a resistance member (e.g., ball plunger or flexure) on a surface of the control base and the control knob surface facing the control base can include a plurality of detents such that the control knob can rotate until the resistance member reaches a detent that can temporarily prevent rotation of the control knob. Note that the resistance member and plurality of detents can be switched to be on the other of the surface of the control knob or control base. The tactile feedback can be provided to the operator when a resistance member reaches a detent. After reaching a first detent, the operator can then further rotate the control knob. The control knob can continue to rotate until the resistance member reaches a second detent. The distance between the plurality of detents can correspond to a predetermined amount of fluid volume. In some embodiments, the distance between the plurality of detents can be the rotational distance or arc distance of rotation of the portion of the syringe body mount for the resistance member to move from one detent to another. Thus, when an operator rotates the control knob such that the resistance member moves from one detent to another, this can signal that a specific predetermined amount of fluid has been dispersed from the syringe.

The tactile feedback can let the syringe operator know each time they have dispensed a predetermined amount of fluid volume from the syringe. In some embodiments, the predetermined amount of fluid volume can be at least about 0.5 microliters, at least about 1 microliter, at least about 5 microliters, at least about 25 microliters, or at least about 50 microliters. In some embodiments, the predetermined amount of fluid volume can be at most about 250 microliters, at most about 100 microliters, at most about 50 microliters, at most about 25 microliters, at most about 10 microliters, or at most about 5 microliters. In some embodiments, the predetermined amount of fluid volume can be about 0.5-5 microliters, about 0.5-1.5 microliters, or about 1 microliter. In some embodiments, the tactile feedback mechanism of the syringe can provide tactile feedback for every microliter dispersed. As explained above, the tactile feedback mechanisms can allow rotation of the control knob to stop at each detent (e.g., at each microliter dispersion), but be easy to overcome to continue the rotation.

In some embodiments, one full revolution of a portion of the syringe body mount (e.g., the control knob) can deliver a second predetermined amount of fluid volume. In some embodiments, the second predetermined amount of fluid volume per revolution of a portion of the syringe body mount (e.g., the control knob) can be at least about 1 microliter, at least about 4 microliters, at least about 5 microliters, at least about 10 microliters, at least about 25 microliters, at least about 50 microliters, at least about 100 microliters, or at least about 250 microliters. In some embodiments, the second predetermined amount of fluid volume per revolution of a portion of the syringe body mount (e.g., the control knob) can be at most about 1000 microliters, at most about 500 microliters, at most about 250 microliters, at most about 100 microliters, at most about 50 microliters, or at most about 25 microliters. In some embodiments, the second predetermined amount of fluid volume per revolution of a portion of the syringe body mount (e.g., the control knob) can be about 1-10 microliters, about 2-8 microliters, about 4-5 microliters, or about 4 microliters.

In some embodiments, the control knob can be easy to grip while rotating. In some embodiments, the control knob (e.g., control knob casing) can have plurality of grooves 30 for gripping the control knob. In some embodiments, the grooves are shallow. In some embodiments, the grooves run along the longitudinal direction of the syringe. In some embodiments, the control knob has at least about 4, at least about 5, at least about 10, at least about 15, at least about 20, at least about 24, or at least about 25 grooves along the circumferential exterior surface of the control knob. In some embodiments, the control knob has a knurled surface for gripping the control knob.

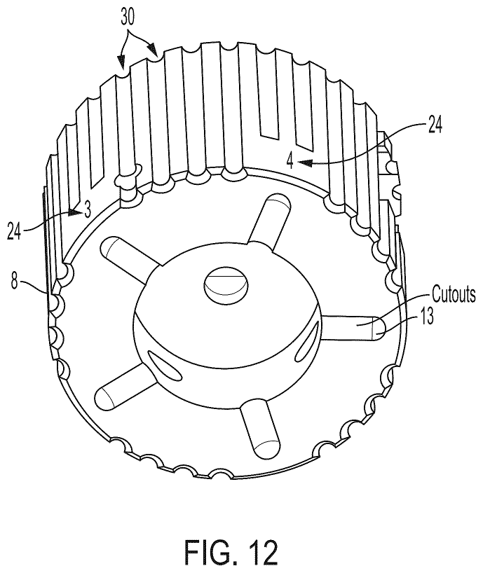

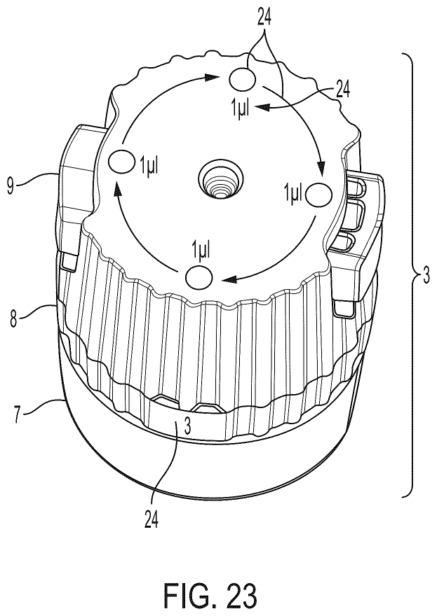

In some embodiments, the syringe body mount can be configured to provide visual feedback to an operator of the syringe when a predetermined amount of fluid volume is dispersed from the syringe. In some embodiments, the control knob or control base can have a plurality of visual indicators (e.g., visual indicators 24 shown in FIGS. 2A, 20A-B, 23, 24A). In some embodiments, the plurality of visual indicators can be molded in or printed on the surface of the control knob or control base. In some embodiments, the visual indicators can be numbers or graphics (e.g., positioning and/or directional graphics). In some embodiments, the numbers can correspond to each predetermined amount of fluid volume dispersed. In other words, the visual indicators can allow the operator to know how much fluid volume has been dispersed (e.g., 1 microliter designations shown in FIG. 23). In some embodiments, the visual indicators match up with the tactile feedback for each predetermined amount of fluid volume dispersed (as illustrated in FIG. 12). In other words, the detents of the control knob or control base can align with the visual indicators. In some embodiments, the control knob or control base can have visual indicator numbers starting with 0 and increase until a full revolution. For example, a full revolution of the control knob can start with 0 and end with 0. When the predetermined amount of fluid volume dispersed is 1 microliter, the control knob (for example) can include visual indicators 0, 1, 2, and 3 for a 4 microliter delivery per revolution of the control knob. In some embodiments, the top of the control knob can have visual indicators 24 as shown in FIG. 23. In some embodiments, the visual indicators can be directional graphics (e.g., arrows) indicating which direction the control knob can be rotated.

In some embodiment, the control base or control knob can have a single visual indicator (e.g., visual indicator 31 shown in FIGS. 2A and 20A) that can provide the operator of the syringe visual feedback that a defined volume has been dispensed when the control knob is rotated. In some embodiments, the single visual indicator can match up with the tactile feedback for each predetermined amount of fluid volume dispersed. In other words, when a predetermined amount of fluid has been dispersed (i.e, when a resistance element is matched with, fit with, or resting in a detent), the first visual indicator (e.g., number visual indicator) of a control knob or control base is aligned with a second visual indicator (e.g., a line or other marking or graphic as shown as 31 in FIGS. 2A and 20A) on the other of one of the control knob or control base. As such, a visual indicator on the control knob can match up or align with a visual indicator of the control base when a predetermined amount of fluid has been dispersed (i.e, when a resistance element is matched with, fit with, or resting in a detent). In some embodiments, the syringes can be designed and such that assembly of the syringes can allow for correct positioning/alignment of the visual indicators and the tactile feedback mechanism (e.g., resistance member and detents).

In some embodiments, the syringes disclosed herein can be single-use syringes. In some embodiments, the syringes can be packaged as single-use. In some embodiments, the shelf life of the syringes disclosed herein can be at least about 6 months, at least about 12 months, at least about 24 months, or at least about 5 years. In some embodiments, the syringes disclosed herein can be sterilized. In some embodiments, the sterilization process may not cause more than minimal discoloration of the syringe body. In some embodiments, the syringes disclosed herein may not need to be able to be re-sterilized.

For the purpose of clarity and a concise description, features are described herein as part of the same or separate embodiments; however, it will be appreciated that the scope of the disclosure includes embodiments having combinations of all or some of the features described.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference to “about” a value or parameter herein includes (and describes) variations that are directed to that value or parameter per se. For example, description referring to “about X” includes description of “X”. In addition, reference to phrases “less than”, “greater than”, “at most”, “at least”, “less than or equal to”, “greater than or equal to”, or other similar phrases followed by a string of values or parameters is meant to apply the phrase to each value or parameter in the string of values or parameters.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The above description is presented to enable a person skilled in the art to make and use the disclosure, and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

1. A syringe comprising:
a syringe body having a proximal end and a distal end;
a threaded plunger, wherein a portion of the threaded plunger is within the syringe body;
a syringe body mount connected to the proximal end of the syringe body, wherein the syringe body mount comprises:
a control base fixed to the proximal end of the syringe body; and
a control knob rotatably connected to the control base, wherein the control knob is configured to engage with threads of the threaded plunger such that rotation of the control knob with respect to the control base causes the threaded plunger to move longitudinally in the syringe body,
wherein the syringe body mount is configured to provide tactile feedback to an operator of the syringe when a predetermined amount of fluid volume is dispersed from the syringe.

2. The syringe of claim 1, wherein the control knob comprises a plunger disengagement mechanism that disengages the control knob from the threads of the threaded plunger such that the threaded plunger is configured to move longitudinally in the syringe body without rotation of the control knob.

3. The syringe of claim 2, wherein the plunger disengagement mechanism comprises at least one depressible button.

4. The syringe of claim 3, wherein the control knob comprises a spring configured to cause the at least one depressible button to engage with the threads of the threaded plunger and disengage with the threads of the threaded plunger when the at least one depressible button is depressed.

5. The syringe of claim 3, wherein the plunger disengagement mechanism comprises two depressible buttons.

6. The syringe of claim 5, wherein the control knob comprises an O-ring configured to cause the two depressible buttons to engage with the threads of the threaded plunger and disengage with the threads of the threaded plunger when the two depressible buttons are depressed.

7. The syringe of claim 1, wherein the control knob is configured to disengage with the threads of the threaded plunger such that the threaded plunger is configured to move longitudinally in the syringe body without rotation of the control knob.

8. The syringe of claim 2, wherein the control knob is configured to disengage with the threads of the threaded plunger such that the threaded plunger is configured to move longitudinally in the syringe body via pulling or pushing on a plunger handle on a proximal end of the threaded plunger.

9. The syringe of claim 1, wherein the control knob is configured to engage with the threads of the threaded plunger such that clockwise rotation of the control knob with respect to the control base causes the threaded plunger to move longitudinally towards the distal end of the syringe body and counterclockwise rotation of the control knob with respect to the control base causes the threaded plunger to move longitudinally towards the proximal end of the syringe body.

10. The syringe of claim 1, wherein the control base comprises a ball plunger and the control knob comprises a plurality of detents such that as the control knob rotates, the ball plunger is depressed between the control base and control knob until each detent where the ball plunger fits into the detent providing tactile feedback to the operator.

11. The syringe of claim 10, wherein distance between the plurality of detents corresponds to the predetermined amount of fluid volume.

12. The syringe of claim 1, wherein the control base comprises a flexure and the control knob comprises a plurality of detents such that as the control knob rotates, the flexure experiences resistance until each detent where the flexure relaxes providing tactile feedback to the operator.

13. The syringe of claim 12, wherein distance between the plurality of detents corresponds to the predetermined amount of fluid volume.

14. The syringe of claim 1, wherein the distal end of the syringe body comprises a luer lock connector.

15. The syringe of claim 1, further comprising a plunger handle on a proximal end of the threaded plunger and a plunger tip on a distal end of the threaded plunger.

16. The syringe of claim 1, wherein the threaded plunger is a left-hand threaded plunger.

17. The syringe of claim 1, wherein the predetermined amount of fluid volume is 1 microliter.

18. The syringe of claim 1, wherein the syringe body mount is configured to provide audio or visual feedback to an operator of the syringe for a predetermined amount of fluid volume dispersed from the syringe.

19. The syringe of claim 1, wherein the syringe body has a working volume of 450-550 microliters.

20. The syringe of claim 1, wherein the threaded plunger does not rotate while moving longitudinally in the syringe body.

21. The syringe of claim 1, wherein the distal end of the syringe is connected to a cannula.

22. A method of treating a subject having a disease or condition comprising use of the syringe of claim 1 to deliver a cell suspension to the subject.

23. The method of claim 22, wherein the disease or condition is a neurodegenerative disease.

24. The method of claim 22, wherein the disease or condition is a Lewy body disease (LBD).

25. The method of claim 22, wherein the disease or condition is Parkinson's disease.

26. The method of claim 22, wherein the cell suspension comprises stem cells or cells that have been differentiated from stem cells.

27. The method of claim 22, wherein the cell suspension comprises neurally differentiated cells.

28. The method of claim 22, wherein the cell suspension is delivered to the brain tissue of the subject.

29. The method of claim 22, wherein the cell suspension is delivered to the striatum, optionally the putamen, of the subject.

30. The method of claim 22, wherein the subject is a human.

31. The method of claim 22, wherein a cannula is connected to the syringe to deliver the cell suspension to the subject.

32. The syringe of claim 15, further comprising a syringe tip comprising a recess configured to receive at least a portion of the syringe body and a channel configured to receive at least a portion of the plunger tip.

33. The syringe of claim 32, wherein the channel comprises a first proximal portion and a second distal portion and the first proximal portion is configured to receive at least a portion of the plunger tip.
